(19) United States
(12) Patent Application Publication
(10) Pub. No.: US 2005/0202465 A1
(43) Pub. Date: Sep. 15, 2005

(54) Thymidylate synthase gene and metastasis

(75) Inventors: Tian-Li Wang, Baltimore, MD (US); Luis Diaz, Eldridge, MD (US); Christoph Lengauer, Columbia, MD (US); Victor Velculescu, Dayton, MD (US); Kenneth W. Kinzler, Bel Air, MD (US); Bert Vogelstein, Baltimore, MD (US)

Correspondence Address: BANNER & WITCOFF, 1001 G STREET N W, SUITE 1100, WASHINGTON, DC, 20001, US

(73) Assignee: The Johns Hopkins University, Baltimore, MD (US)

(21) Appl. No.: 10/996,070

(22) Filed: Nov. 24, 2004

Related U.S. Application Data
(60) Provisional application No. 60/541,942, filed on Feb. 6, 2004.

(51) Int. Cl. C12Q 1/68; G06F 19/00; G01N 33/48; G01N 33/50

(52) U.S. Cl. 435/6; 702/20

(57) ABSTRACT

Thymidylate synthase (TYMS) gene amplification was observed in 23% of 31 5-FU resistant liver metastases, while no amplification was observed in metastases of patients that had not been treated with 5-FU. Patients with metastases containing TYMS amplification had a substantially shorter median survival (329 days) than those without amplification (1021 days, p<0.01). Genetic amplification of TYMS has important implications for the management of colorectal cancer patients with recurrent disease.

This application claims priority to provisional Application Ser. No. 60/541,942 filed Feb. 6, 2004.

This invention was made using funds from National Institutes of Health grants CA43460, CA57345, and CA62924. The U.S. government therefore retains certain rights in the invention

### TECHNICAL FIELD OF THE INVENTION

This invention is related to the area of cancer. In particular, it relates to diagnostics, therapeutics, and drug discovery for cancer.

### BACKGROUND OF THE INVENTION

Since its introduction over four decades ago, 5-FU has become a staple of treatment for many cancers. In particular, it is the mainstay of chemotherapeutic regimens for colorectal cancers, both in metastatic and adjuvant settings (1). Metabolites of 5-FU and other fluoropyrimidines irreversibly inhibit thymidylate synthase (TYMS, Online Mendelian Inheritance in Man (OMIM) reference number 188350), the enzyme normally responsible for conversion of deoxyuridine monophosphate to deoxythymidine monophosphate (2). As this process generates the sole de novo source of thymidylate, an essential precursor to DNA synthesis, inhibition of TYMS leads to DNA damage and blocks DNA replication and repair. In addition to its effects on DNA, metabolites of 5-FU can be incorporated into RNA, thereby disrupting normal RNA processing and function.

Although many colorectal cancer patients initially respond to 5-FU based therapies, most develop recurrences and are usually treated with 5-FU in combination with other drugs such as oxaliplatin (3, 4) or irinotecan (5). A subset of patients respond to such therapy, but in a variable and unpredictable manner. Despite significant research on the effects of 5-FU on cancer cells in vitro (2, 6-9), the molecular mechanisms underlying the development of 5-FU resistance in patients remain largely unknown. High TYMS protein or mRNA levels in tumors as determined by immunohistochemistry or RT-PCR has tended to be associated with a worse response to 5-FU in patients (10, 11). However, some reports have shown the opposite, i.e., that patients with high TYMS protein expression have improved outcome compared to those with low expression when treated with 5-FU (12). Additionally, measurement of TYMS protein expression in primary tumors does not aid in predicting outcome or response to 5-FU at sites of metastatic disease (13, 14). Alterations in levels of enzymes affecting 5-FU metabolism, including thymidine phosphorylase (TP) and dihydropyrimidine dehydrogenase (DPD), have also been postulated to affect 5-FU resistance. Overexpression of TP protein has been reported to increase sensitivity to 5-FU (15), while elevated levels of DPD mRNA have been associated with resistance (16). However, these correlations are also controversial, as some studies have shown that increased levels of TP mRNA were found in tumors that were less likely to respond to 5-FU (17), while others have reported that DPD and TP protein levels have no effect on patient survival (18).

Though levels of protein and RNA expression can provide clues to causal events during tumorigenesis, gene expression is difficult to measure accurately for technical reasons and may be affected by complex regulatory circuits specific to each tumor's environment. In contrast, genetic alterations can provide unambiguous information about pathogenetic mechanisms. For example, genetic alterations of the p53 gene provided critical clues to its pathogenic role that were not anticipated from prior measurements of p53 protein expression levels (19). Similarly, genetic mutations and gene amplification of the BCR/ABL gene in patients refractory to therapy with Gleevec have provided unique insights into the mechanisms underlying resistance to this tyrosine kinase inhibitor (20, 21). Unfortunately, previous genetic studies on 5-FU resistance have been limited to analyses of the development of chemoresistance in vitro (2, 6-9) or to a small number of patient case reports (22, 23).

Based on above genetic precedents and the lack of a systematic study of 5-FU resistance in human cancer, we have undertaken a comprehensive genomic analysis of 5-FU resistance in colorectal cancer using digital karyotyping (DK) (24). DK permits high resolution analyses of copy number alterations on a genome-wide scale. The approach involves isolation and high-throughput analysis of short (21 bp) sequence tags from ˜800,000 specific loci distributed throughout the genome. Analysis of sequence tag densities in sliding windows throughout each chromosome allows identification of potential amplifications and deletions at high resolution. Our analysis represents a systematic genetic examination of resistance to 5-FU in vivo, convincingly identifying TYMS gene amplification as a major determinant of 5-FU chemoresistance of human cancers.

There is a continuing need in the art to refine the management of cancer patients. Tools for stratifying patients to make better therapeutic decisions could improve anti-tumor response and decrease unnecessary side effects.

### SUMMARY OF THE INVENTION

A first embodiment of the invention is a method for categorizing patients who have been treated with 5-FU. A copy number of a gene encoding thymidylate synthase is determined in tumor tissue of a patient who has been treated with 5-FU. The patient is assigned to a first category if the patient has a hyperdiploid copy number. The patient is assigned to a second category if the patient does not have a hyperdiploid copy number.

A second embodiment of the invention is a method of screening agents for ability to treat 5-FU resistant tumors. A test agent is contacted with (1) first human cells having a hyperdiploid copy number of thymidylate synthase; and (2) second human cells having a diploid copy number of thymidylate synthase. A parameter is determined for each of the first and second human cells. The parameter is selected from the group consisting of: apoptosis, growth rate, viability, and colony number. The test agent is identified as a candidate for treating 5-FU resistant tumors if the test agent preferentially increases apoptosis or decreases growth rate, viability, or colony number in the first human cells relative to the second human cells.

A third embodiment is a method of assessing agents for ability to treat 5-FU resistant tumors. A test agent is contacted with (1) a first population of humans having a tumor with a hyperdiploid copy number of thymidylate synthase gene, and (2) a second population of humans having a tumor with a diploid copy number of thymidylate synthase gene. A parameter for each of the first and second populations is determined. The parameter is selected from the group consisting of: tumor regression, tumor marker decrease, and clinical condition. The test agent is identified as a candidate for treating 5-FU resistant tumors if the agent preferentially or equally increases tumor regression, tumor marker decrease, or improves clinical condition in the first human population relative to the second human population.

These and other embodiments which will be apparent to those of skill in the art upon reading the specification provide the art with methods for stratification, prognosis, therapy, and drug screening pertaining to cancer patients that have been treated with 5-fluorouracil.

### BRIEF DESCRIPTION OF THE DRAWINGS

FIG. 1. Overlapping regions of amplification on chromosome 18p identified by digital karyotyping. Bitmap views comprised of 18,431 pixels representing tag density values at the chromosomal position of each virtual tag on chromosome 18. Yellow regions indicate tag densities that were not amplified, while black regions represent areas with genomic tag densities indicating amplification. Genomic tag densities were determined as described in Experimental Procedures and had maximal values of 10 and 6 copies per diploid genome for the amplifications in FU-M2 and FU-M4, respectively. Genes present within overlapping amplified regions are indicated below on a high resolution map. Only TYMS and rTS were entirely contained within the regions that were amplified in both FU-M2 and FU-M4.

FIGS. 2A-2D. Quantitative PCR analysis of genomic DNA from colorectal metastases. Quantitive PCR analysis of TYMS (right curves) and LINE element control (left curves) performed on genomic DNA from colorectal cancer metastases (FIG. 2A) FU-M2 and (FIG. 2B) FU-M4, and (FIG. 2C) normal (non-tumor) DNA. (FIG. 2D) Differences in threshold cycle numbers between LINE element and TYMS confirm that TYMS is present at increased gene copy numbers in colorectal metastases.

FIGS. 3A-3C. TYMS amplification in 5-FU resistant cancers. FISH analysis of interphase nuclei from (FIG. 3A) 5-FU resistant colorectal cancer metastasis to the liver, and matched (FIG. 3B) colorectal adenoma obtained prior to 5-FU treatment and (FIG. 3C) colorectal cancer from a patient with FAP after 5-FU neoadjuvant therapy. Nuclei are visualized with 4′,6′-diamidino-2phenylindole stain (DAPI) (blue), TYMS probe (located on chromosome 18 position 0.8-1.0 Mb from the telomere) is visualized using FITC-avidin (green), and chromosome 18 control probe (located on chromosome 18 position 13.0-13.2 Mb from the telomere) is visualized using TRITC-conjugated antibodies (red). Increased TYMS gene copy number was only observed in 5-FU resistant cancers.

FIG. 4. Five year survival curve for patients with and without TYMS amplification.

### DETAILED DESCRIPTION OF THE INVENTION

The inventors have developed a test for determining appropriate treatment and prognosis for patients with tumors that have previously been treated with 5-FU. A subpopulation of such patients have genomic amplification of a region of chromosome 18p that contains the thymidylate synthase gene. Patients that have this genomic amplification in their tumors have a significantly shorter life expectancy than patients that do not. Moreover, the tumors of patients with this genomic amplification are refractory to the beneficial effects of 5-FU. Thus this subpopulation of patients should not be treated with 5-FU in order to eliminate unnecessary toxicity.

Copy number of the gene encoding thymidylate synthase can be determined by any technique known in the art. Copy number can, for example, be determined using quantitative PCR, digital karyotyping, FISH (fluorescence in situ hybridization) or Southern blotting. Reliable and sensitive quantitative methods are preferred. A hyperdiploid copy number (3 or more copies per diploid genome) can be determined with respect to any other gene or genes which are present at the diploid number of copies. Because many cancer cells are aneuploid, the copy number of the TYMS gene may be compared to the copy number of another gene on chromosome 18 or chromosome 18p. Such a comparison will distinguish between tumors where all of chromosome 18 or 18p is present in a hyperdiploid number and tumors which have a hyperdiploid complement of chromosome 18 or 18p. The local genomic amplification may be quite pronounced, with copy numbers of the TYMS gene greater than 6, 8, 10, 12, or 14 per diploid genome in tumor tissue of a patient who has been treated with 5-FU.

Stratification of patients is useful for determining both a therapeutic plan and life expentancy. Patients may be assigned to two or more groups based on the copy number of the TYMS gene. A patient with a hyperdiploid number should not be treated with 5-FU or other therapeutic agent which inhibits TS. A patient without the genomic amplification can be treated with 5-FU.

Life expectancy predictions can be used to make a number of decisions. Such predictions can influence choices of therapy, as well as whether to have certain treatments or not. For example, the choice to undergo or forego elective surgery, experimental therapy, or palliative therapy may be influenced by life expectancy. Patients may choose to retire or take disability leave based on life expectancy predictions. Patients may decide to use hospice care based on such predictions. Patients may establish legal documents to govern their care if they are incapacitated based on life expectancy predictions. Patients may write or change a will based on life expectancy predictions. Thus such predictions can have multiple ramifications for the patient and the family, beyond the issue of use of 5-FU. Clinicians may choose not to deliver life expectancy predictions directly to patients or their families, but may instead make suggestions about one or more of these ramifications based on the life expectancy prediction.

Tumor tissue is often heterogeneous, comprising cells of a number of different types, including both neoplastic and non-neoplastic cells. In order to make determination of copy number more accurate, steps can be taken to obtain a cell sample which is predominantly neoplastic. Tumor epithelial cells of the metastatic tumor tissue are desirably used for the cell sample. Such cells can be purified away from other cells by any technique known in the art, including immunopurification. One such immunopurification employs anti-BerEP4 immunomagnetic beads available from Dynal, Oslo, Norway. Any other technique known in the art for separating neoplastic from non-neoplastic cells can be used. Primary tumors, recurrent tumors, or metastatic tumors can be tested according to the present invention.

5-FU is used inter alia to treat cancers of the colon, breast, stomach, head and neck, anus, skin, and pancreas. In addition, it is used to treat metastases including lung and liver metastases. Metastases from any of these organs or others can be tested in the classification method of the present invention.

As described above, a subset of metastases are resistant to 5-FU due to genomic amplification. Cells with such amplification can be used to discover new drugs which might be used on such resistant tumors. Test agents may include substances or compositions which are synthetic, semi-synthetic, or natural products. Test agents may be novel compounds or compositions or known compounds or products. A test agent which has potential as a useful drug for treating 5-FU resistant metastases can be identified by observing effects on human cells which have a hyperdiploid copy number of thymidylate synthase gene and human cells which have a a diploid copy number of thymidylate synthase gene. The effect can be observed on apoptosis, growth rate, viability, or colony number. Those of skill in the art know how to measure these parameters. A test agent can be identified as a candidate for treating 5-FU resistant tumors if the agent preferentially or equally increases apoptosis or decreases growth rate, viability, or colony number in the 5-FU resistant human cells relative to the 5-FU sensitive human cells. The cells used in the test may be tumor cells or cells which are genetically engineered so that one of them contains the desired amplification. The two types of cells used can be taken from the same patient or different patients. Similar tests can be conducted on patients stratified by the presence or absence of amplification of TYMS in their tumors. The effect of a test agent can be observed by any criterion known in the art. These include tumor regression, tumor marker decrease, and clinical condition including life span.

The results described below in the examples demonstrate a significant association between treatment with 5-FU, amplification of the TYMS gene, and survival following surgical excision of metastatic lesions. These observations have significant implications for basic and clinical aspects of human cancer.

Drug resistance is a major cause of treatment failure and death in cancer patients. But the basis for drug resistance in such tumors is generally unknown. There have been numerous reports of mechanisms underlying the development of drug resistance in cell culture systems. For example, expression of multi-drug resistance (MDR) genes can confer resistance to drugs in vitro (31, 32), but the relationship of such expression to the development of resistance in vivo remains conjectural. Similarly, the dihydrofolate reductase (DHFR) gene is commonly found to be amplified after treatment of cultured cells with methotrexate (33), but there are only a few case reports of gene amplification occurring in the tumors of cancer patients after exposure to conventional chemotherapeutic agents in vivo (22, 34, 35).

There are two examples of gene amplification developing in patients treated with targeted therapies, and both are informative with respect to 5-FU. The first involves androgen receptor mutations in prostate cancer patients treated with anti-androgens (36). The second involves genetic alterations (often amplification) of the BCR/ABL fusion gene in chronic myelogenous leukemia (CML) patients treated with Gleevec (20, 21). Gleevec inhibits several tyrosine kinases in addition to BCR/ABL (37). However, the specific genetic alterations of BCR/ABL that develop during treatment unambiguously point to the BCR/ABL gene as the central drug target in CML. Similarly, many potential mechanisms of action of 5-FU have been suggested (8, 38, 39). But the amplification of the TYMS gene following 5-FU treatment in vivo provides compelling evidence that TYMS is a major target of this drug in human cancer patients.

The fact that TYMS amplification was only observed in patients after treatment with 5-FU suggests that the cancers must pass through a bottleneck that effectively kills the vast majority of cancer cells (those without TYMS gene amplification) in these patients. Our observations, coupled with the experimental demonstration that engineered overexpression of TYMS in cultured cells can cause 5-FU resistance (40), provide compelling evidence that TYMS amplification is responsible for a significant fraction of 5-FU resistance.

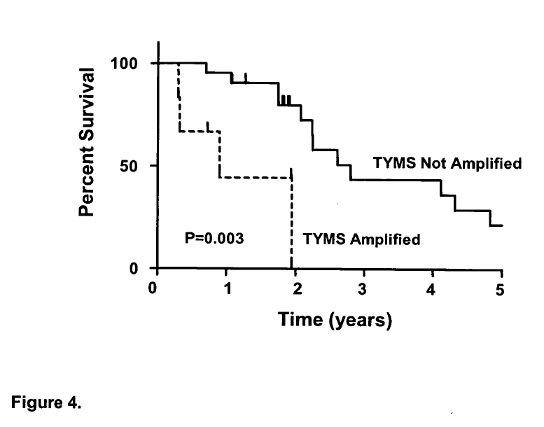

The considerably worse survival of patients with TYMS amplification compared to similar patients without TYMS gene amplification (Table 2 and FIG. 4) is consistent with the conclusion that the amplification of this gene is responsible for 5-FU resistance. Though the reasons for the reduced survival are not known with certainty, patients whose tumors recur following metastectomy are generally treated with regimens containing 5-FU. In our patient population, the majority of patients received 5-FU alone or in combination with other chemotherapeutic agents following removal of metastases (data not shown). The 5-FU component of such regimens would not likely be of benefit in patients with TYMS gene amplification but would be expected to cause the same degree of systemic toxicity observed in patients without TYMS gene amplification, potentially explaining the worse survival of these patients.

In addition to TYMS amplification, it is possible that other genetic mechanisms of resistance are present in patients with clinical resistance to 5-FU. It is important to note that not all patients without TYMS amplification had longer survival times, and in fact several patients had extremely short survival periods following surgical resection. Such mechanisms of resistance could include genetic modification of other members of the TYMS pathway, including TP or DPD, or candidate genes within previously described loci affected by 5-FU in vitro (8, 41).

Though larger, prospective studies will be important to confirm the present findings, our results have clear implications for the management of colorectal cancer patients. In particular, our data suggest that recurrences in patients whose biopsies show TYMS gene amplification should not be treated with 5-FU. Many of the newer second-line therapies undergoing clinical trials involve combinations of 5-FU with other agents. In patients with TYMS gene amplification, the 5-FU would likely add toxicity without efficacy. TYMS gene amplification is straightforward to detect using the probes and methods described in this paper, and can be performed on routinely fixed and paraffin-embedded samples. In addition to eliminating the 5-FU from regimens that would ordinarily use it, these results should stimulate efforts to develop compounds that specifically target cancers with amplified TYMS genes (42) and provide an ideal subset of patients in which to test such agents.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

### Materials and Methods

Tissue Samples

Tissue samples, including normal tissues, primary tumors, and metastases were obtained from colorectal cancer patients undergoing surgery at the Johns Hopkins Hospital between 1990 and 2002. A diagnosis of colorectal cancer was established by histological examination of surgical specimens and clinical information was retrospectively retrieved from patient records. Acquisition of tissue specimens and examination of clinical records was approved by an institutional review board and was performed in accordance with HIPAA regulations. Metastatic samples were obtained from complete resections, debulking, or biopsies of metastatic lesions.

Tumor Cell Purification of Liver Metastases

Tumor cells were purified from liver metastases as previously described (25). Briefly, tissues were obtained immediately following surgical removal and digested with 1 mg/ml collagenase for 1 hour at 37° C. Single cell suspensions were obtained by sequential filtering through nylon mesh of 400 μm, 50 μm, and 25 μm. Epithelial cells were isolated by binding to anti-BerEP4 immunomagnetic beads (Dynal, Oslo, Norway) and the purified cells were immediately frozen at −80° C.

Digital Karyotyping

Digital karyotyping libraries were constructed as previously described (24). Briefly, genomic DNA was isolated using a DNeasy kit (Qiagen, Chatsworth, Calif.). For each sample, 1 μg of genomic DNA was sequentially digested with mapping enzyme SacI (New England Biolabs, Beverly, Mass.), ligated to 20-40 ng of biotinylated linkers (Integrated DNA Technologies, Coralville, Iowa), and digested with the fragmenting enzyme NlaIII (New England Biolabs, Beverly, Mass.). DNA fragments containing biotinylated linkers were isolated by binding to streptavidin-coated magnetic beads (Dynal, Oslo, Norway). Captured DNA fragments were ligated to linkers containing MmeI recognition sites, and tags were released with MmeI (New England Biolabs, Beverly, Mass.). Tags were self-ligated to form ditags which were then further ligated to form concatemers and cloned into pZero (Invitrogen, Carlsbad, Calif.). Clones were sequenced using Big Dye terminators (ABI, Foster City, Calif.) and analyzed using a 384 capillary automated sequencing apparatus (Spectrumedix, State College, Pa.) or with a 96 capillary ABI 3700 instrument at Agencourt Biosciences (Beverly, Mass.). Digital karyotyping sequence files were trimmed using Phred sequence analysis software (CodonCode, Mass.) and 21 bp genomic tags were extracted using the SAGE2000 software package. Tags were matched to the human genome (UCSC human genome assembly, June 2002 freeze) and tag densities were evaluated using the digital karyotyping software package. Genomic densities were calculated as the ratio of experimental tags to the number of virtual tags present in a fixed window. Sliding windows of sizes ranging from 100 to 300 virtual tags were used to identify regions of increased and decreased genomic density. Chromosomal regions were considered to contain an amplification if maximal genomic densities were >6 genome copies per diploid genome. Digital karyotyping protocols and software for extraction and analysis of genomic tags are available at http://www.digitalkaryotyping.org.

Quantitative PCR

Genome content differences between metastatic tumors and normal liver cells were determined by quantitative real-time PCR using an iCycler apparatus (Bio-Rad, Hercules, Calif.) as previously described (24). DNA content was normalized to that of Line-1, a repetitive element for which copy numbers per haploid genome are similar among all human cells. PCR primers with the following sequences

| | |
|---|---|
| TYMS-F: | |
| 5′-TTTTCGAAGAATCCTGAGCTTTG-3′ | (SEQ ID NO: 1) |
| and | |
| TYMS-R | |
| 5′-CACTCTCGATCTGTGCAAGAGAA-3′ | (SEQ ID NO: 2) |

were used to amplify a portion of the TYMS gene located at chromosome 18 position 988801 bp-989046 bp (UCSC human genome assembly, June 2002 freeze).

PCR reactions for each sample were performed in triplicate and threshold cycle numbers were calculated using iCycler software v2.3 (Bio-Rad Laboratories, Hercules, Calif.).

FISH

Formalin-fixed paraffin-embedded tissue array sections 4 um in thickness were analyzed by FISH as previously described (26, 27). BAC clone RP11-806L2 (located on chromosome 18 position 0.8-1.0 Mb from the telomere) and RP1-151D11 (located on chromosome 18 position 13.0-13.2 Mb from the telomere) were obtained from Bacpac Resources (Children's Hospital Oakland, Calif.) and used as probes for the TYMS gene and a reference region on chromosome 18, respectively. RP1-806L2 and RP1-151D11 were labeled by nick translation with biotin-dUTP and digoxigenin-dUTP, respectively. To detect biotin-labeled and digoxigenin-labeled signals, slides were first incubated with FITC-avidin (Vector, Burlingame, Calif.) and an anti-digoxigenin mouse antibody (Roche, Indianapolis, Ind.). The slides were subsequently incubated with a biotinylated anti-avidin antibody (Vector, Burlingame, Calif.) and TRITC-conjugated rabbit anti-mouse antibody (Sigma, St. Louis, Mo.), then finally incubated with FITC-avidin and TRITC-conjugated goat anti-rabbit antibody (Sigma). Slides were counterstained with 4′,6′-diamidino-2-phenylindole stain (DAPI) (Sigma, Burlingame, Calif.).

FISH signals were evaluated with a Nikon fluorescence microscope E800 by two individuals who were blinded to the treatment history of each patient. Separate narrow band pass filters were used for the detection of TRITC, FITC, and DAPI signals. Using 40× objective lens, approximately one hundred tumor cells were examined for each specimen and the number of fluorescent signals within tumor cells from the TYMS gene BAC probe and chromosome 18 reference BAC probe were recorded. Amplification of the TYMS gene was defined as a ratio of TYMS BAC probe signals to chromosome 18 reference BAC probe signals 2:1 or greater.

Statistical Analysis

Overall survival was calculated from the date of the surgical excision of the metastasis to the date of death or last follow-up and computed by Kaplan-Meier method. Data were censored when patients were lost to follow-up.

### EXAMPLE 2

### Digital Karyotyping of Colorectal Cancer Metastases

DK was used to evaluate genomic DNA from liver metastases of four different colorectal cancer patients that had previously received 5-FU based adjuvant chemotherapy (FU-M1-4). As controls, two liver metastases from colorectal cancer patients that had not previously received 5-FU (M1-2) were also analyzed. In each case, tumor epithelial cells were immunopurified from the metastases using antibody-conjugated magnetic beads (25). This purification was useful to obtain DNA templates that were free of significant contamination from non-neoplastic cells within the metastatic lesions.

A total of ˜200,000 genomic tags were obtained from each sample, permitting analysis of loci spaced at an average distance of ˜30 kb throughout the genome. Computation of genomic tag densities identified distinct sub-chromosomal regions of amplification and deletion on several chromosomes. All of the alterations occurred in individual tumors with the exception of a region of amplification on chromosome 18. This amplification, located at 18p11.32, was observed in two of the four 5-FU resistant metastases (FU-M2 and FU-M4), but not in the two metastases from untreated patients, suggesting that this region could be related to 5-FU resistance.

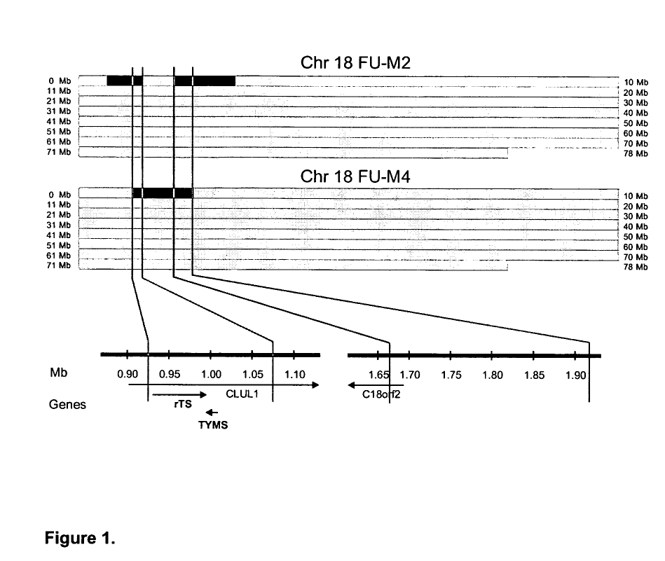

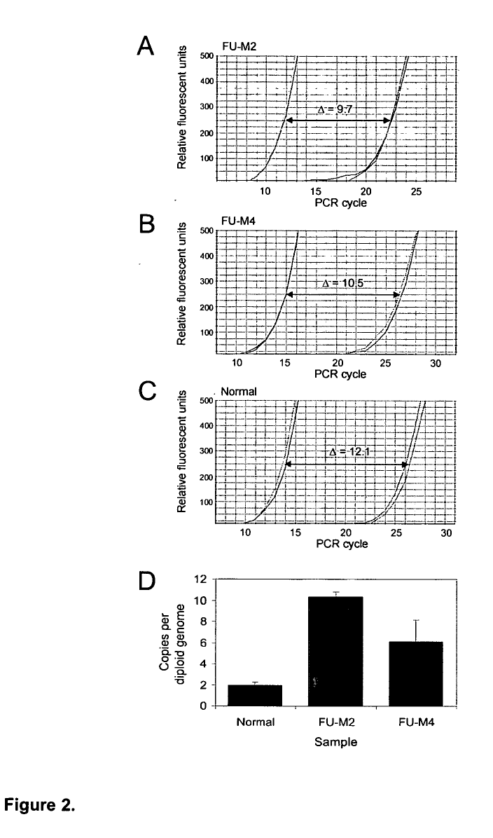

In tumor FU-M2, two separate but closely spaced amplicons were identified at 18p11.32, while a single continuous amplicon was found in tumor FU-M4 in the same region. Detailed analyses of these amplicons showed two common regions of amplification, one 0.92-1.06 Mb from the telomere and the other 1.66-1.92 Mb from the telomere (FIG. 1). Examination of genome databases identified two genes that were completely contained within the first region, TYMS and the HSRTSβgene (rTS), while no known or predicted genes were present in the second. Amplification of TYMS was of particular interest because (i) the region containing this gene had a higher tag density within the overlapping amplicons; (ii) TYMS expression has been correlated with 5-FU resistance in some studies (10, 11), and (iii) TYMS amplification has been documented to develop in cancer cell lines that became resistant to 5-FU after exposure to this drug in vitro (28, 29). Quantitative PCR analyses of genomic DNA using primers specific to the TYMS locus confirmed that TYMS was amplified to levels of 10 and 6 gene copies per diploid genome in FU-M2 and FU-M4, respectively (FIG. 2).

### EXAMPLE 3

### FISH Analysis of TYMS Amplification

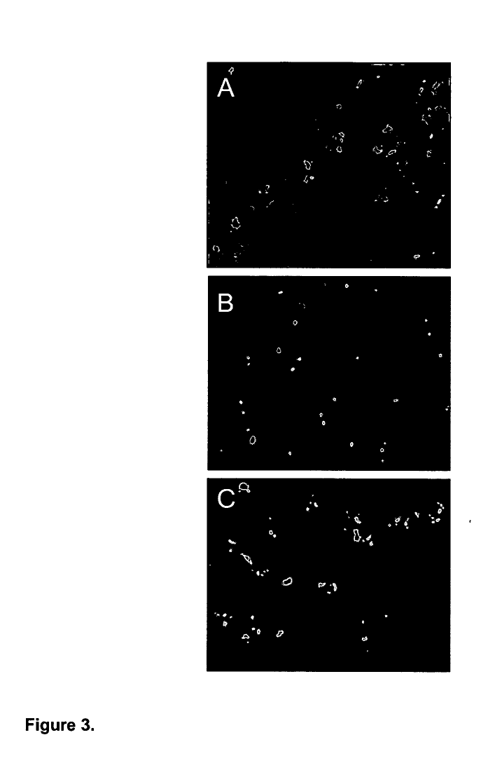

To further evaluate the role of TYMS in 5-FU resistance, we analyzed TYMS gene copy number using dual-color fluorescence in situ hybridization (FISH). A total of 89 colorectal cancers embedded in tissue microarrays were assessed. These comprised 53 metastases derived from liver, lung and brain tissues, including the four metastases originally analyzed by DK, and 36 primary colorectal cancers. Thirty one of the analyzed lesions were from patients that had received 5-FU therapy prior to tumor resection. Biotinylated DNA from a bacterial artificial chromosome (BAC) containing the TYMS gene was used as probe and sections were co-hybridized with digoxigenin-labeled DNA from a BAC containing sequences from 18p11.21, 12 Mb closer to the centromere. Two probes from the same chromosome are necessary to distinguish chromosome duplications from true amplification events, the latter involving relatively small amplicons (30). Using FISH, multiple copies of the TYMS gene were detected in interphase nuclei in seven lesions, including the two metastases previously detected by DK (Table 1, example in FIGS. 3A and 3C). All seven lesions were derived from patients who had been treated with 5-FU: six were metastatic lesions of patients who had previously been treated with 5-FU based therapy, while one was a primary colorectal cancer from a Familial Adenomatous Polyposis (FAP) patient who had been treated with 5-FU prior to colectomy. In contrast, none of the 58 cancers from patients that had not been treated with 5-FU showed increased copies of the TYMS gene (Table 1, p<0.001, Chi-squared test). To examine the temporal relationship between TYMS amplification and 5-FU treatment, we analyzed a primary colorectal cancer from a patient that later developed metastases with TYMS amplification. This primary cancer was removed prior to the initiation of 5-FU therapy and did not contain amplified TYMS genes when studied by FISH. Similarly, TYMS amplification in the FAP patient was only present in the resected cancer and was not observed in adenoma tissue obtained prior to 5-FU treatment (FIG. 3B). This observation demonstrates that detectable TYMS amplification occurred only after administration of 5-FU in these patients.

TABLE 1
Prevalence of TYMS amplification in
colorectal cancers

Prior treatment

| TYMS Status | 5-FU | No 5-FU | Total |
|---|---|---|---|
| Amplified | 7 | 0 | 7 |
| Not amplified | 24 | 58 | 82 |
| Total | 31 | 58 | 89 |

### EXAMPLE 4

### TYMS Amplification and Survival

The results described above show that TYMS amplification is exclusively found in cancer lesions of patients who had been treated with 5-FU. These data are consistent with the idea that the exposure to 5-FU had selected for cells with amplified TYMS genes, and that such cells would be resistant to 5-FU treatment. A corollary of this idea is that such patients would fare worse than those without TYMS amplification, as post-surgical therapy of patients with metastatic cancers often involves retreatment with 5-FU plus other agents (see Introduction). To evaluate this possibility, we compared the survival of patients with metastatic lesions that had previously been treated with 5-FU, segregated according to TYMS amplification status. Although the average age, stage at initial diagnosis, and metastasis size and location were similar between patients with and without increased TYMS gene copies, the median overall survival following surgical removal of metastases among patients with TYMS gene amplification was 329 days, as compared with 1021 days for patients without TYMS amplification (Table 2, p<0.01, t-Test). Using a proportional hazard model, patients with TYMS amplification showed relative risk of death that was 3.5 fold higher (relative risk 1.06-11.4, 95% confidence interval, p<0.05) than patients without TYMS amplification. These differences were also significant in Kaplan-Meier analyses (FIG. 4). In particular, this analysis showed that no patient with TYMS amplification has survived longer than two years while over a quarter of the patients without TYMS amplification survived over 4 years (FIG. 4, p<0.01 Logrank test).

TABLE 2
Clinical characteristics of 5-FU treated patients with
and without TYMS amplification

| | | | | | | | Median survival after |
|---|---|---|---|---|---|---|---|
| | TYMS status in | Previous | Age | Stage at | Metastasis | Metastasis | metastasis removal |
| Patient Group | metastasis | treatment | (years) | diagnosis | size (cm) | location | (survival range) |
| Group A (n = 6) | Amplified | 5-FU | 65 | I - 0% | 2.2 | Liver - 67% | 329 days |
| | | | | II - 0% | | Lung - 0% | (109-708 days) |
| | | | | III - 33% | | Brain - 33% | |
| | | | | IV - 50% | | | |
| | | | | ND - 17% | | | |
| Group B (n = 21) | Not amplified | 5-FU | 63 | 1 - 5% | 3.2 | Liver—80% | 1021 days |
| | | | | II - 10% | | Lung - 10% | (255-3790 days) |
| | | | | III - 29% | | Brain - 10% | |
| | | | | IV - 43% | | | |
| | | | | ND - 14% | | | |

### REFERENCES

The disclosure of each reference cited is expressly incorporated herein.
1. Moertel, C. G. (1994) N Engl J Med 330, 1136-42.
2. Longley, D. B., Harkin, D. P. & Johnston, P. G. (2003) Nat Rev Cancer 3, 330-8.
3. Giacchetti, S., Perpoint, B., Zidani, R., Le Bail, N., Faggiuolo, R., Focan, C., Chollet, P., Llory, J. F., Letourneau, Y., Coudert, B., Bertheaut-Cvitkovic, F., Larregain-Fournier, D., Le Rol, A., Walter, S., Adam, R., Misset, J. L. & Levi, F. (2000) J Clin Oncol 18, 136-47.
4. de Gramont, A., Figer, A., Seymour, M., Homerin, M., Hmissi, A., Cassidy, J., Boni, C., Cortes-Funes, H., Cervantes, A., Freyer, G., Papamichael, D., Le Bail, N., Louvet, C., Hendler, D., de Braud, F., Wilson, C., Morvan, F. & Bonetti, A. (2000) J Clin Oncol 18, 2938-47.
5. Saltz, L. B., Cox, J. V., Blanke, C., Rosen, L. S., Fehrenbacher, L., Moore, M. J., Maroun, J. A., Ackland, S. P., Locker, P. K., Pirotta, N., Elfring, G. L. & Miller, L. L. (2000) N Engl J Med 343, 905-14.
6. Peters, G. J., Backus, H. H., Freemantle, S., van Triest, B., Codacci-Pisanelli, G., van der Wilt, C. L., Smid, K., Lunec, J., Calvert, A. H., Marsh, S., McLeod, H. L., Bloemena, E., Meijer, S., Jansen, G., van Groeningen, C. J. & Pinedo, H. M. (2002) Biochim Biophys Acta 1587, 194-205.
7. Plasencia, C., Rooney, P. H., Taron, M., Martinez-Balibrea, E., McLeod, H. L. & Abad, A. (2003) Int J Oncol 22, 945-53.
8. Maxwell, P. J., Longley, D. B., Latif, T., Boyer, J., Allen, W., Lynch, M., McDermiott, U., Harkin, D. P., Allegra, C. J. & Johnston, P. G. (2003) Cancer Res 63, 4602-6.
9. Wang, W., Marsh, S., Cassidy, J. & McLeod, H. L. (2001) Cancer Res 61, 5505-10.
10. Johnston, P. G., Lenz, H. J., Leichman, C. G., Danenberg, K. D., Allegra, C. J., Danenberg, P. V. & Leichman, L. (1995) Cancer Res 55, 1407-12.
11. Lenz, H. J., Hayashi, K., Salonga, D., Danenberg, K. D., Danenberg, P. V., Metzger, R., Banerjee, D., Bertino, J. R., Groshen, S., Leichman, L. P. & Leichman, C. G. (1998) Clin Cancer Res 4, 1243-50.
12. Edler, D., Glimelius, B., Hallstrom, M., Jakobsen, A., Johnston, P. G., Magnusson, I., Ragnhammar, P. & Blomgren, H. (2002) J Clin Oncol 20, 1721-8.
13. Findlay, M. P., Cunningham, D., Morgan, G., Clinton, S., Hardcastle, A. & Aherne, G. W. (1997) Br J Cancer 75, 903-9.
14. Johnston, P. G., Benson, A. B., 3rd, Catalano, P., Rao, M. S., O'Dwyer, P. J. & Allegra, C. J. (2003) J Clin Oncol 21, 815-9.
15. Evrard, A., Cuq, P., Robert, B., Vian, L., Pelegrin, A. & Cano, J. P. (1999) Int J Cancer 80, 465-70.
16. Salonga, D., Danenberg, K. D., Johnson, M., Metzger, R., Groshen, S., Tsao-Wei, D. D., Lenz, H. J., Leichman, C. G., Leichman, L., Diasio, R. B. & Danenberg, P. V. (2000) Clin Cancer Res 6, 1322-7.
17. Metzger, R., Danenberg, K., Leichman, C. G., Salonga, D., Schwartz, E. L., Wadler, S., Lenz, H. J., Groshen, S., Leichman, L. & Danenberg, P. V. (1998) Clin Cancer Res 4, 2371-6.
18. Ikeguchi, M., Makino, M. & Kaibara, N. (2002) Langenbecks Arch Surg 387, 240-5.
19. Baker, S. J., Fearon, E. R., Nigro, J. M., Hamilton, S. R., Preisinger, A. C., Jessup, J. M., vanTuinen, P., Ledbetter, D. H., Barker, D. F., Nakamura, Y., White, R. & Vogelstein, B. (1989) Science 244, 217-21.
20. Gorre, M. E., Mohammed, M., Ellwood, K., Hsu, N., Paquette, R., Rao, P. N. & Sawyers, C. L. (2001) Science 293, 876-80.
21. Shah, N. P., Nicoll, J. M., Nagar, B., Gorre, M. E., Paquette, R. L., Kuriyan, J. & Sawyers, C. L. (2002) Cancer Cell 2, 117-25.
22. Clark, J. L., Berger, S. H., Mittelman, A. & Berger, F. G. (1987) Cancer Treat Rep 71, 261-5.
23. Gorlick, R., Metzger, R., Danenberg, K. D., Salonga, D., Miles, J. S., Longo, G. S., Fu, J., Banerjee, D., Klimstra, D., Jhanwar, S., Danenberg, P. V., Kemeny, N. & Bertino, J. R. (1998) J Clin Oncol 16, 1465-9.
24. Wang, T. L., Maierhofer, C., Speicher, M. R., Lengauer, C., Vogelstein, B., Kinzler, K. W. & Velculescu, V. E. (2002) Proc Natl Acad Sci USA 99, 16156-61.
25. Saha, S., Bardelli, A., Buckhaults, P., Velculescu, V. E., Rago, C., St Croix, B., Romans, K. E., Choti, M. A., Lengauer, C., Kinzler, K. W. & Vogelstein, B. (2001) Science 294, 1343-6.
26. Lengauer, C., Kinzler, K. W. & Vogelstein, B. (1997) Nature 386, 623-7.
27. Bardelli, A., Saha, S., Sager, J., Romans, K. E., Xin, B., Markowitz, S. D., Lengauer, C., Velculescu, V. E., Kinzler, K. W. & Vogelstein, B. (2003) Clin Cancer Res in press.
28. Jenh, C. H., Geyer, P. K., Baskin, F. & Johnson, L. F. (1985) Mol Pharmacol 28, 80-5.
29. Berger, S. H., Jenh, C. H., Johnson, L. F. & Berger, F. G. (1985) Mol Pharmacol 28, 461-7.
30. Brodeur, G. M. & Hogarty, M. D. (1998) in The genetic basis of human cancer, eds. Kinzler, K. W. & Vogelstein, B. (McGraw-Hill, New York), Vol. 1, pp. 161-179.
31. Kartner, N., Riordan, J. R. & Ling, V. (1983) Science 221, 1285-8.
32. Gros, P., Ben Neriah, Y. B., Croop, J. M. & Housman, D. E. (1986) Nature 323, 728-31.
33. Schimke, R. T. (1984) Cell 37, 705-13.
34. Horns, R. C., Jr., Dower, W. J. & Schimke, R. T. (1984) J Clin Oncol 2, 2-7.
35. Carman, M. D., Schornagel, J. H., Rivest, R. S., Srimatkandada, S., Portlock, C. S., Duffy, T. & Bertino, J. R. (1984) J Clin Oncol 2, 16-20.
36. Koivisto, P., Visakorpi, T. & Kallioniemi, O. P. (1996) Scand J Clin Lab Invest Suppl 226, 57-63.
37. Druker, B. J. (2002) Cancer Cell 1, 31-6.
38. Scherf, U., Ross, D. T., Waltham, M., Smith, L. H., Lee, J. K., Tanabe, L., Kohn, K. W., Reinhold, W. C., Myers, T. G., Andrews, D. T., Scudiero, D. A., Eisen, M. B., Sausville, E. A., Pommier, Y., Botstein, D., Brown, P. O. & Weinstein, J. N. (2000) Nat Genet 24, 236-44.
39. Hwang, P. M., Bunz, F., Yu, J., Rago, C., Chan, T. A., Murphy, M. P., Kelso, G. F., Smith, R. A., Kinzler, K. W. & Vogelstein, B. (2001) Nat Med 7, 1111-7.
40. Saga, Y., Suzuki, M., Mizukami, H., Kohno, T., Takei, Y., Fukushima, M. & Ozawa, K. (2003) Int J Cancer 106, 324-6.
41. Rooney, P. H., Stevenson, D. A., Marsh, S., Johnston, P. G., Haites, N. E., Cassidy, J. & McLeod, H. L. (1998) Cancer Res 58, 5042-5.
42. Neuteboom, S. T., Karjian, P. L., Boyer, C. R., Beryt, M., Pegram, M., Wahl, G. M. & Shepard, H. M. (2002) Mol Cancer Ther 1, 377-84.

1. A method for categorizing patients who have been treated with 5-FU, comprising:
determining a copy number of a gene encoding thymidylate synthase in tumor tissue of a patient who has been treated with 5-FU;
assigning the patient to a first category if the patient has a hyperdiploid copy number relative to one or more other genes located on chromosome 18, and assigning the patient to a second category if the patient does not have a hyperdiploid copy number relative to one or more other genes located on chromosome 18.

2. The method of claim 1 wherein the tumor tissue is metastatic tumor tissue.

3. The method of claim 1 wherein a hyperdiploid number is assigned if the gene encoding thymidylate synthase has an increased copy number relative to one or more other genes on chromosome 18p.

4. The method of claim 1 further comprising recommending that a patient assigned to the first category not be treated with 5-FU

5. The method of claim 1 further comprising recommending that a patient assigned to the second category be treated with 5-FU.

6. The method of claim 1 further comprising:
predicting a reduced life expectancy for a patient assigned to the first category relative to patients in the second category.

7. The method of claim 1 further comprising:
predicting a longer life expectancy for a patient assigned to the second category relative to patients in the first category.

8. The method of claim 1 wherein the copy number is determined in tumor epithelial cells of the metastatic tumor tissue.

9. The method of claim 8 wherein the tumor epithelial cells are immunopurified.

10. The method of claim 1 wherein the copy number is determined by digital karyotyping.

11. The method of claim 1 wherein the copy number is >3 per diploid genome.

12. The method of claim 1 wherein the copy number is determined by fluorescence in in situ hybridization.

13. The method of claim 1 wherein the patient had a primary colorectal tumor.

14. The method of claim 1 wherein the tumor tissue is from a lung metastasis.

15. The method of claim 1 wherein the tumor tissue is from a liver metastasis.

16. A method of screening agents for ability to treat 5-FU resistant tumors comprising:
contacting a test agent with (1) first human cells having a hyperdiploid copy number of thymidylate synthase gene relative to one or more other genes located on chromosome 18; and (2) second human cells having a diploid copy number of thymidylate synthase gene;
determining a parameter for each of the first and second human cells, said parameter selected from the group consisting of: apoptosis, growth rate, viability, and colony number;
identifying the test agent as a candidate for treating 5-FU resistant tumors if the agent preferentially increases apoptosis or decreases growth rate, viability, or colony number in the first human cells relative to the second human cells.

17. The method of claim 16 wherein the first and second human cells are tumor cells.

18. The method of claim 16 wherein the first and second human cells are from the same patient.

19. A method of assessing agents for ability to treat 5-FU resistant tumors comprising:
contacting a test agent with (1) a first population of humans having a tumor with a hyperdiploid copy number of thymidylate synthase gene relative to one or more other genes located on chromosome 18; and (2) a second population of humans having a tumor with a diploid copy number of thymidylate synthase gene;
determining a parameter for each of the first and second populations, said parameter selected from the group consisting of: tumor regression, tumor marker decrease, and clinical condition;
identifying the test agent as a candidate for treating 5-FU resistant tumors if the agent preferentially or equally increases tumor regression, tumor marker decrease, or improves clinical condition in the first human population relative to the second human population.

20. The method of claim 19 wherein the tumor is a metastatic tumor.


SEQUENCE LISTING (2 sequences)

SEQ ID NO: 1 - DNA, 23 bp, Homo sapiens
ttttcgaagaatcctgagctttg

SEQ ID NO: 2 - DNA, 23 bp, Homo sapiens
cactctcgatctgtgcaagagaa